(19) United States
(12) Patent Application Publication
(10) Pub. No.: US 2006/0064136 A1
(43) Pub. Date: Mar. 23, 2006

(54) Method and apparatus for facilitating patient alert in implantable medical devices

(75) Inventor: Li Wang, White Bear Township, MN (US)

Correspondence Address: MEDTRONIC, INC., 710 MEDTRONIC PARK, MINNEAPOLIS, MN, 55432-9924, US

(73) Assignee: Medtronic, Inc.

(21) Appl. No.: 10/947,803

(22) Filed: Sep. 23, 2004

(51) Int. Cl. A61N 1/08

(52) U.S. Cl. 607/27

(57) ABSTRACT

An implantable medical device system capable of generating a patient alert message for display on an external patient communicator is provided. The patient alert message notifies the patient that a patient alert condition has been detected; indicates what type of alert condition was detected, and instructs the patient regarding what action should be taken. The patient alert message may additionally provide an indication of the severity of the patient alert condition. The IMD system includes an IMD capable of sensing signals for detecting multiple patient alert conditions and generating a patient alert signal. The IMD system further includes an external patient communicator capable of receiving patient alert data from the IMD. Upon receipt of the patient alert data, the patient communicator displays a patient alert message including patient instructions which typically indicate appropriate medical personnel contact information.

### FIELD OF THE INVENTION

The present invention relates generally to implantable medical devices and more particularly to an implantable medical device system and method for generating a patient alert.

### BACKGROUND OF THE INVENTION

A variety of implantable medical devices (IMDs) are commercially available for monitoring physiological signals and for delivering therapies, such as cardiac pacemakers and defibrillators, hemodynamic monitors, drug delivery devices, insulin monitors and pumps, and neuromuscular stimulators. These devices may detect clinically serious or life-threatening conditions related to physiological events or conditions. Such devices are also typically enabled to perform self-diagnostic tests or otherwise monitor device performance issues, such as battery life-expectancy, electrical lead impedance, frequency of delivered therapies, and so on. The advanced capabilities of IMDs in detecting physiological or device-related conditions that may warrant clinical attention or even emergency care has motivated the addition of real time patient warning features to IMD systems.

A real-time warning alerts the patient that a condition requiring medical attention has been detected. Patients may be alerted to the detection of a condition requiring medical attention through sensory stimulation delivered by the IMD such as audible tones or low level muscle stimulation. For example, audible patient alarms have been proposed to be incorporated in an IMD to warn a patient of battery depletion as generally disclosed in U.S. Pat. Nos. 4,345,603 and 4,488,555, incorporated herein by reference. Similarly, the application of low energy stimulation to electrodes on or near the IMD to provide a perceptible tingling or twitching has been generally proposed in U.S. Pat. Nos. 4,140,131, and 5,076,272, both of which are incorporated herein by reference, and also in the above-incorporated '603 patent. Use of an audible alarm incorporated into an implantable cardioverter/defibrillator to warn the patient of impending delivery of a cardioversion shock is generally disclosed, for example, in U.S. Pat. No. 4,210,149, incorporated herein by reference.

Audible sounds, which may include voiced statements, may be emitted by an IMD, for example, to warn a patient of battery energy depletion or imminent delivery of a therapy as generally disclosed in U.S. Pat. No. 6,067,473 issued to Greeninger et al., incorporated herein by reference in its entirety. Acoustic or visual display messages to alert or warn a patient may be incorporated in external medical devices as generally disclosed in U.S. Pat. No. 5,285,792, issued to Sjoquist et al., incorporated herein by reference in its entirety.

With a growing number of monitoring and detection features available in implantable medical devices, patient alarms may be generated for a number of different physiological or device-related triggering conditions. The patient may be aware of a patient alarm or alert but unaware of the severity or type of condition that has triggered a sensory patient alert. As a result, the patient may be unsure as to the most appropriate action and the urgency of any required action he or she should take to address the potentially serious or even life-threatening condition. Furthermore, when a patient alarm is implemented as an auditory signal, a patient that has difficulty hearing or is in a noisy environment may be unable to hear the alarm.

### BRIEF SUMMARY OF THE INVENTION

The present invention provides in one embodiment, an IMD system capable of generating a patient alert message for display on an external patient communicator. The patient alert message notifies the patient that a patient alert condition has been detected; indicates what type of alert condition was detected, and instructs the patient regarding what action should be taken. The patient alert message may additionally provide an indication of the severity of the patient alert condition. The IMD system includes an IMD capable of detecting multiple patient alert triggering conditions and generating a patient alert signal in response to an alert condition. The IMD system further includes an external patient communicator device capable of receiving the patient alert signal from the IMD. The IMD is enabled to detect one or more alert triggering conditions relating to sensed physiological signals or monitored device performance parameters. Upon detecting an alert triggering condition, based on predefined trigger condition criteria, a patient alert signal is generated. In one embodiment the patient alert signal is a wireless communication signal containing patient alert data for transmission to the patient communicator. In another embodiment, the patient alert signal is sensory stimulus delivered to the patient to notify the patient of the presence of a patient alert condition. The patient is previously advised to initiate a communication session between the IMD and the patient communicator upon perceiving a sensory alert signal to allow patient alert data to be transferred to the patient communicator and a patient alert message to be displayed.

In one embodiment, the patient communicator is a dedicated device for receiving patient alert data through a communication link with the IMD upon detection of a patient alert triggering condition by the IMD. The patient communicator responds to an alert signal by displaying an alert message including the alert condition information and patient instructions. The patient instructions may include phone numbers of the appropriate medical personnel to contact regarding the triggering condition. Patient instructions and other alert information are displayed in accordance with programmable data stored in the patient communicator or IMD memory.

In another embodiment, the patient communicator is implemented in an external medical device (EMD) such as a patient programmer or home monitor. The EMD includes the patient communicator functions for receiving patient alert telemetry from the IMD and displaying an alert message in addition to other programming or monitoring functions, which may include transferring data, including patient alert data, to a central database for remote patient monitoring.

### BRIEF DESCRIPTION OF THE DRAWINGS

FIG. 1 illustrates an IMD system including a patient communicator unit in accordance with the present invention, for communicating patient alert information to a patient.

FIG. 2 is a block diagram of typical functional components of an IMD, such as the IMD shown in FIG. 1.

FIG. 3 is a functional block diagram of an IMD in communication with a patient communicator for transferring patient alert data for display by the patient communicator.

FIG. 4 is an illustration of an exemplary patient communicator alert message display.

FIG. 5 is a flow chart summarizing steps included in a patient alert method.

### DETAILED DESCRIPTION OF THE PREFERRED EMBODIMENTS

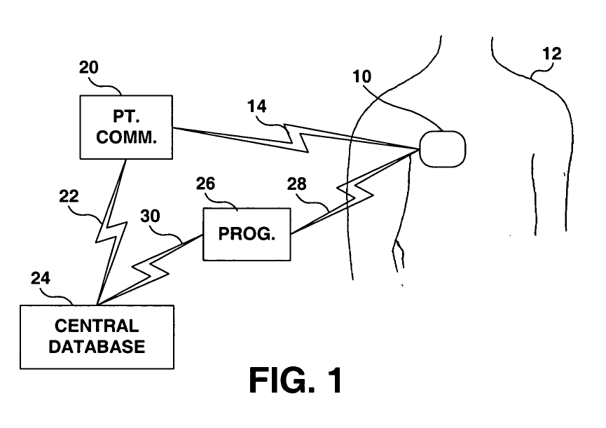

FIG. 1 illustrates an IMD system including a patient communicator unit in accordance with the present invention, for communicating patient alert information to a patient. IMD 10 is shown implanted in a patient 12. The simplified illustration of IMD 10 may represent a variety of IMDs such as cardiac pacemakers, implantable cardioverter defibrillators, hemodynamic monitors, ECG recorders, drug delivery devices, insulin monitors or pumps, or neuromuscular stimulators. IMD 10 may be coupled to one or more leads or fluid delivery catheters. Leads may be used for carrying electrodes or physiological sensors used for monitoring one or more physiological conditions and delivering electrical stimulation therapies. IMD 10 may alternatively be embodied as a leadless device wherein sensors or electrodes are incorporated in or on the housing of IMD 10. Examples of leadless monitoring devices are generally disclosed in U.S. Pat. No. 5,404,877 issued to Nolan et al., and U.S. Pat. No. 5,987,352 issued to Klein et al, both of which patents are incorporated herein by reference in their entirety.

IMD 10 is provided with an antenna and associated circuitry, as will be described below, for establishing a communication link 14 with patient communicator 20. Patient communicator 20 may be embodied as dedicated device for performing patient alert functions as will be described herein. Alternatively, patient communicator 20 may be implemented as a component of a home programmer or monitoring unit 26 which includes other IMD programming and interrogation functions as well as within other devices such as a PDA, personal computer pager or the like. Programmer and home monitoring units for use with an IMD are known in the art.

As will be described in greater detail herein, patient alert information can be transferred to the patient communicator 20 from IMD 10 through communication link 14. Communication link 14 may be a unidirectional communication link allowing data to be transferred from IMD 10 to patient communicator 20. In other embodiments, patient communicator 20 may be enabled to transfer data to IMD 10 via a bidirectional communication link, for example to confirm receipt of a complete patient alert data transmission. When patient communicator 20 is implemented as a component of programmer or monitoring unit 26, patient alert data may be transferred to programmer 26 via bidirectional communication link 28.

Patient communicator 20 may optionally be adapted to communicate with a central database 24 to allow transfer of patient alert data received from IMD 10 to the central database 24. A central database may be an Internet-based or other networked database used for remote patient monitoring. Patient communicator 20 may transfer data via a communication link 22, which may be established via the Internet, a local area network, a wide area network, a telecommunications network or other appropriate communications network and may be a wireless communication link.

Alternatively, programmer or home monitoring unit 26 may receive patient alert data from IMD 10 or patient communicator 20 and transfer the patient alert data to central database 24 using a communication link 30. In this way, programmer/home monitor 26 may be used to transfer patient alert information along with other remote monitoring data. Examples of remote monitoring systems are generally disclosed in U.S. Pat. No. 6,599,250 issued to Webb et al., U.S. Pat. No. 6,442,433 issued to Linberg, and U.S. Pat. No. 6,574,511 issued to Lee, U.S. Pat. No. 6,480,745 issued to Nelson et al., U.S. Pat. No. 6,418,346 issued to Nelson et al., and U.S. Pat. No. 6,250,309 issued to Krichen et al., all of which patents are incorporated herein by reference in their entirety.

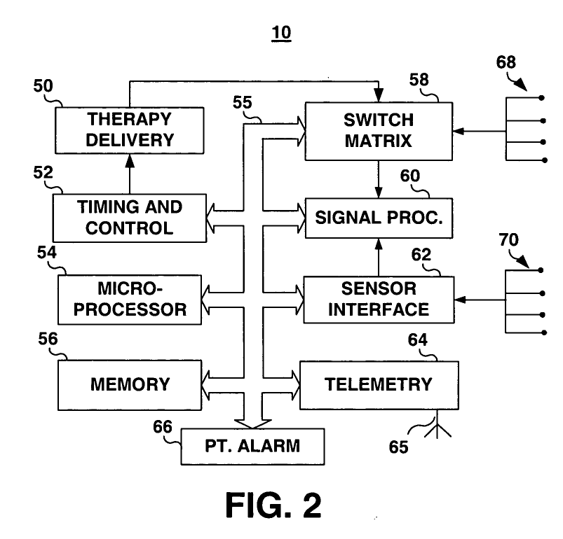

FIG. 2 is a block diagram of typical functional components of an IMD, such as IMD 10 shown in FIG. 1. IMD 10 generally includes timing and control circuitry 52 and an operating system that may employ microprocessor 54 or a digital state machine for timing sensing and therapy delivery functions in accordance with a programmed operating mode. Microprocessor 54 and associated memory 56 are coupled to the various components of IMD 10 via a data/address bus 55. IMD 10 may include therapy delivery unit 50 for delivering a therapy, such as an electrical stimulation or drug therapy, under the control of timing and control 52. In the case of electrical stimulation therapies, such as cardiac stimulation therapies, therapy delivery unit 50 is typically coupled to two or more electrodes 68 via a switch matrix 58. Switch matrix 58 is used for selecting which electrodes and corresponding polarities are used for delivering electrical stimulation pulses.

Electrodes 68 may also be used for sensing electrical signals within the body, such as cardiac signals, or for measuring impedance. In the case of cardiac stimulation devices, cardiac electrical signals are sensed for determining when an electrical stimulation therapy is needed and in controlling the timing of stimulation pulses. In some embodiments, detection of a need for therapy delivery, such as a defibrillation therapy, is a patient alert condition, causing IMD 10 to issue a patient alert signal.

Electrodes used for sensing and electrodes used for stimulation may be selected via switch matrix 58. When used for sensing, electrodes 68 are coupled to signal processing circuitry 60 via switch matrix 58. Signal processor 60 includes sense amplifiers and may include other signal conditioning circuitry and an analog to digital converter. Electrical signals may then be used by microprocessor 54 for detecting physiological events, such as detecting and discriminating cardiac arrhythmias. In some embodiments, cardiac arrhythmia detection may be classified as a patient alert condition, which would cause IMD 10 to issue a patient alert signal. In other embodiments, electrodes 68 may be used for measuring impedance signals for monitoring edema, respiration or heart chamber volume. Any of these signals may be used to detect a change indicating a worsening pathologic condition, which may trigger a patient alert. Impedance signals can also be used for monitoring lead performance and detecting lead-related problems as is known in the art.

IMD 10 may additionally or alternatively be coupled to one or more physiological sensors 70. Such sensors may include pressure sensors, accelerometers, flow sensors, blood chemistry sensors, activity sensors or other physiological sensors known for use with IMDs. Sensors 70 are coupled to IMD 10 via a sensor interface 62 which provides sensor signals to signal processing circuitry 60. Sensor signals are used by microprocessor 54 for detecting physiological events or conditions. For example, IMD 10 may monitor heart wall motion, blood pressure, blood chemistry, respiration, or patient activity. Monitored signals may be used for sensing the need for delivering a therapy under control of the operating system. Physiological events or changes in monitored physiological conditions may be defined as triggering conditions for a patient alert signal to be generated by IMD 10.

The operating system includes associated memory 56 for storing a variety of programmed-in operating mode and parameter values that are used by microprocessor 54. The memory 56 may also be used for storing data compiled from sensed physiological signals and/or relating to device operating history for telemetry out on receipt of a retrieval or interrogation instruction. All of these functions and operations are known in the art, and many are generally employed to store operating commands and data for controlling device operation and for later retrieval to diagnose device function or patient condition. In accordance with the present invention, parameter values or limits defining a number of patient alert trigger conditions may be stored in memory 56 and used by microprocessor 54 in detecting patient alert conditions. Additionally, information to be included in a patient alert message, such as medical personnel contact information, may be stored in memory 56.

IMD 10 further includes telemetry circuitry 64 and antenna 65. Programming commands or data are transmitted during uplink or downlink telemetry between IMD telemetry circuitry 64 and external telemetry circuitry included in a programmer or monitoring unit. In accordance with the present invention, patient alert data is transmitted from IMD 10 via telemetry circuitry 64 and antenna 65 to a patient communicator having associated telemetry circuitry and an antenna for receiving patient alert data. As noted previously, the patient communicator may be implemented in a programmer or home monitoring unit and, as such, not require dedicated telemetry circuitry for receiving patient alert data. Telemetry circuitry 64 and antenna 65 may correspond to telemetry systems known in the art.

In some embodiments, telemetry circuitry may require patient intervention to initiate or enable transfer of patient alert data to a patient communicator. For example, telemetry circuitry 64 may require the use of an external programming head containing an external antenna to be positioned over IMD 10 as generally disclosed in U.S. Pat. No. 5,354,319 issued to Wyborny et al. Telemetry circuitry 64 may require manual “waking up” by the patient to enable data transmission or may require the patient to be within a limited communication range from the patient communicator. In other embodiments, long range telemetry systems may be used allowing patient alert data to be transferred automatically when it is available without intervention by the patient. Long-range telemetry systems are generally disclosed in U.S. Pat. No. 6,482,154 issued to Haubrich et al., incorporated herein by reference in its entirety.

IMD 10 may optionally be equipped with patient alarm circuitry 66 for generating audible tones, a perceptible vibration, muscle stimulation or other sensory stimulation for notifying the patient that a patient alert condition has been detected by IMD 10. Since a sensory alarm may be indiscriminate between patient alert triggering conditions, patient alarm 66 included in IMD 10 is provided as an optional addition to the patient communicator and associated method for displaying the presence of a patient alert message including identifying the type of alert condition and informing the patient of the appropriate action he or she should take. If telemetry circuitry 64 requires patient intervention to initiate transfer of patient alert data from IMD 10 to a patient communicator, a sensory patient alarm 68 is included in IMD 10 to notify the patient that a patient alert data transfer is required. The patient is advised previously to initiate a communication session between the IMD 10 and patient communicator upon perceiving a sensory patient alarm. As such, in some embodiments, the generation of a patient alert signal upon detection of triggering condition causes IMD 10 to generate a sensory patient alarm and prepare for or automatically initiate a patient alert data transmission to the patient communicator.

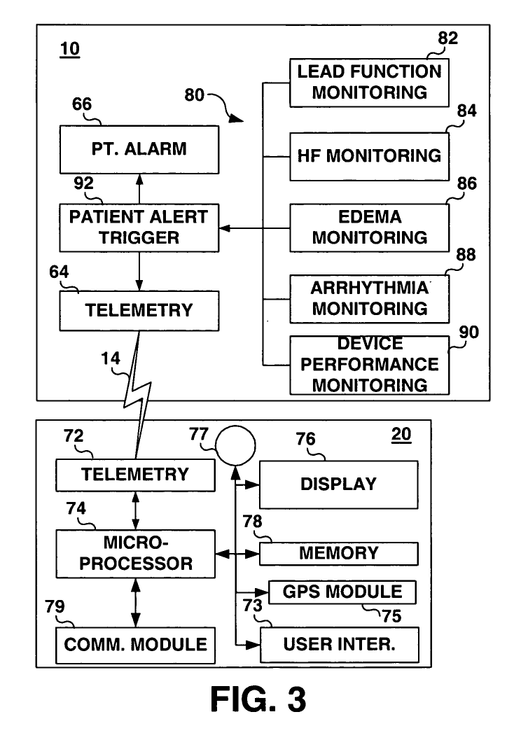

FIG. 3 is a functional block diagram of an IMD in communication with a patient communicator for transferring patient alert data for display by the patient communicator. IMD 10 performs one or more monitoring functions 80, which may include monitoring of device performance and/or monitoring of physiological conditions. In the example shown, monitoring functions 80 include lead function monitoring 82, heart failure monitoring 84, edema monitoring 86, arrhythmia monitoring 88, and device performance monitoring 90.

A patient alert condition may be defined with regard to any of these monitoring functions 80. The operating system of IMD 10 performs a comparative analysis of sensed signals, or parameters derived there from, to determine if predefined alert triggering conditions are present. If a predefined trigger condition is detected, a patient alert trigger signal 92 is generated. In one embodiment, the patient alert trigger signal 92 causes IMD 10 to “wake up” telemetry unit 64 to automatically transfer patient alert data to patient communicator 20 via telemetry link 14. Additionally, the patient alert trigger signal 92 may cause patient alarm 66 to generate sensory stimulation to the patient. Alternatively, patient alert trigger signal 92 causes patient alarm 66 to generate sensory stimulation such that the patient initiates a patient alert data transfer to patient communicator 20.

Patient communicator 20 will typically include a telemetry circuit 72 as described previously for receiving patient alert data from IMD 10. Patient communicator 20 may be a microprocessor-controlled device wherein microprocessor 74 operates with associated memory 78 for generating a message on display 76. The alert message will contain information based on the patient alert data received from IMD 10. As noted previously, components shown to be included in patient communicator 20 may be provided as components in a home programmer or monitoring unit and shared for other programming or patient monitoring functions.

Memory 78 is used alone or in combination with IMD memory 56 (FIG. 2) to store information used in generating a display in response to receiving a patient alert signal. Such information may include recommended patient actions to be performed in response to the patient alert. In particular, appropriate medical personnel contact information may be stored in memory 78 for display in response to an associated patient alert signal. Other information such as prescribed medication recommendations or other instructions tailored to a particular patient may be stored in memory 78 for display upon receipt of an associated patient alert signal. Display information stored in memory 78 may be revised or updated by an authorized care giver by reprogramming stored information using a user interface 73. User-interface 73 which may also be used for clearing, resetting or reviewing previously displayed alert messages.

Display information stored in memory 78 may alternatively be revised or updated with information entered by an authorized user on a centralized programming and monitoring system used for remote patient monitoring. For example, if contact information changes, this change may be entered on a centralized system used for remote patient monitoring and updated in memory 78 upon the next communication session between the centralized system and patient communicator 20. In this way, a clinician may enter updated contact information in one central database 24 (shown in FIG. 1) and the change will be transferred to all patient communicators having communication with the centralized system. In this way, reprogramming of stored information that applies to a number of patients does not require reprogramming information in each individual patient communicator. Moreover, a clinician may make changes to recommended patient actions for a particular patient via reprogramming of information stored in memory 78, either remotely or under direct supervision, as the patient condition changes. Therefore messages displayed on display 76 in response to a particular patient alert signal may change over time in accordance with programmable information stored in memory 78.

Patient communicator 20 may further include a speaker 77 for generating audible sounds to notify the patient that a patient alert message is being displayed. Particularly in the case of automatic transmission of a patient alert signal to patient communicator 20, the patient may be unaware of a displayed patient alert message. In some embodiments, a patient may be made aware of the presence of a displayed patient alert message through a sensory patient alarm 66, audible sounds generated by speaker 77, and/or a flashing display shown on display 76. Numerous alternative methods may be conceived for drawing the patient's attention to the patient alert display which may include generating an electronic mail message, a telephone call, a fax or other communication technologies. Thus patient communicator 20 may include a communications module 79, which may be embodied as a modem, used for placing a telephone call or sending electronic messages to notify the patient or other family members or caregivers of the presence of an alarm message.

Patient communicator 20 may be configured to be a device that is wearable by the patient so that patient alert information received by patient communicator 20 is promptly available for viewing by the patient. Furthermore, patient communicator 20 may optionally include a global positioning system (GPS) module 75 to allow the location of the patient to be determined using GPS technology. GPS capabilities for locating the global location of a patient are particularly beneficial in case of a medical emergency in which the patient is debilitated or unresponsive. A GPS unit may be implemented in patient communicator 20 as generally disclosed in U.S. Pat. No. 5,752,976 issued to Duffin et al., incorporated herein by reference in its entirety.

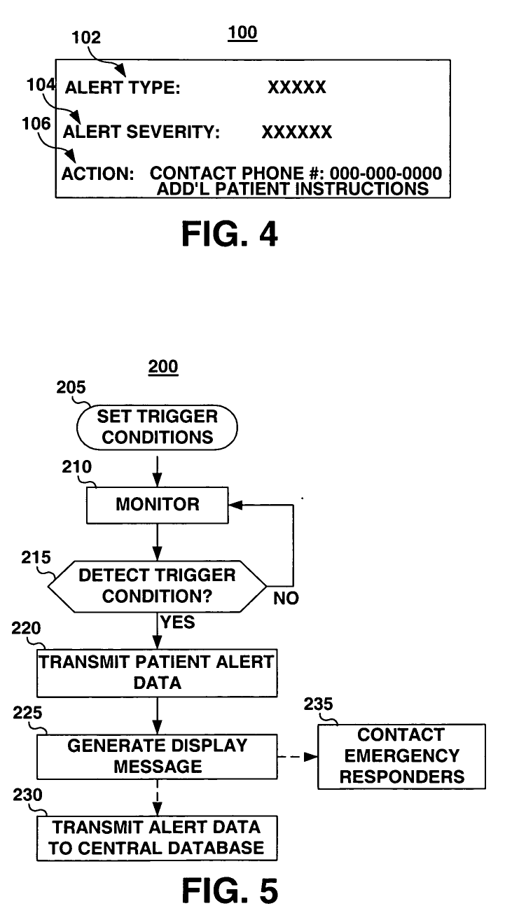

FIG. 4 is an illustration of an exemplary patient communicator alert message display. Display 100 includes a number of fields for communicating to the patient the patient alert data received from an IMD. In field 102, the alert type is indicated. In one embodiment, the display 100 indicates to the patient in field 102 what monitoring function has caused the alert condition. The alert type may be indicated generally according to the monitoring functions enabled. With regard to the exemplary embodiment shown in FIG. 3, the alert condition displayed in field 102 may be specified as relating to lead monitoring, heart failure monitoring, edema monitoring, arrhythmia monitoring, or device performance monitoring. Alternatively, the alert type may be indicated in greater specificity by displaying in field 102 an indication of the monitoring function causing the patient alert and an indication of the particular patient alert trigger condition detected. One or more patient alert triggering conditions may defined relating to each of the IMD monitoring functions. Predefined patient alert triggering conditions may include for example, a high or low lead impedance, a high or low blood pressure, an increase or decrease in lung wetness, a detected and classified arrhythmia, low battery, or any other criteria relating to IMD monitoring functions.

Display 100 may also provide an alert severity field 104 for indicating the urgency of the alert condition. Some patient alert triggering conditions may require medical attention in a few days or even weeks. Other patient alert triggering conditions, such as sudden increase in lung wetness may warrant immediate clinical attention. As such, the severity or urgency of the patient alert condition may be indicated in field 104, thereby indicating to the patient how soon he or she should seek medical attention. A specified interval of time may be displayed within which time the patient should seek medical attention or a general indication of the urgency of the condition may be displayed, e.g. “highly urgent,” “not urgent,” etc.

Display 100 further includes an action field 106 for communicating to the patient an appropriate action to be taken by the patient. The action field 106 will display the contact information, typically a telephone number, for the appropriate medical care provider to contact in regard to the patient alert condition. For example, if an arrhythmia, device or lead-related condition triggers a patient alert, the patient should generally contact an electrophysiology nurse for further instructions or scheduling an appointment. If a heart failure or fluid wetness condition triggers a patient alert, the patient should generally contact a heart failure nurse for further instructions or scheduling. In extremely urgent conditions, the patient may be advised to phone an emergency number. In this way, the most appropriate care provider is contacted first, facilitating a quick response by a clinician to a patient alert condition. Contact information displayed in field 106 may be programmed into the IMD 10 or patient communicator 20 by the medical care provider.

Action field 106 may further include additional patient instructions such as advising the patient to rest, reminding the patient of medication, advising the patient to change the dosage of a prescribed medication or add or remove medications, prepare for receiving a defibrillation shock, requesting the patient initiate a remote monitoring session or any other appropriate action to be taken by or recommended to the patient.

As noted previously, one or more patient alert conditions that cause a patient alert to be triggered are predefined and relate to any monitoring or self-diagnostic function that the IMD is enabled to perform. With further reference to FIG. 3, lead function monitoring 82 typically includes lead impedance measurements performed for detecting a lead problem, such as a conductor failure, poor lead connection, or lead dislodgement. Lead monitoring functions and conditions for triggering a patient alert may be provided, for example, as generally disclosed in U.S. Pat. Nos. 6,317,633 and 6,721,600 issued to Jorgenson et al., hereby incorporated herein by reference in their entirety.

As shown in FIG. 3, a patient alert condition may be defined with regard to lead function monitoring 82 such that if a suspected lead problem is detected, a patient alert trigger 92 is generated. Patient alert data transferred to the patient communicator 20 and formatted for display by patient communicator 20 in the form of a patient alert message may specifies the alert condition as a lead-related condition and may further indicate which lead and what type of lead problem has been diagnosed. The patient alert message may additionally indicate the urgency or severity of the problem. For example, if a lead-related alert condition is detected on a bipolar pacing lead which may be used in a unipolar pacing configuration, the urgency of addressing the condition may be less than if a lead-related alert condition is detected on a defibrillation lead needed for delivering life-saving defibrillation shocks. Displayed patient alert information will include patient instructions for contacting the appropriate medical care provider for addressing the condition.

Patient alert triggering conditions may also be defined relative to heart failure (HF) monitoring 84. HF monitoring may include monitoring one or more physiological signals relating to cardiac hemodynamic functions. HF monitoring may include, for example, monitoring signals relating to blood pressure, blood oxygen saturation, heart wall motion, heart chamber volume, cardiac-related breathing disorders, mechanical restitution, daily activity, heart rate, nighttime heart rate, heart rate variability, or other signals affected by changes in HF status or pathologic myocardial function. Examples of IMDs and methods that may be used for monitoring physiological signals that may relate to heart failure are generally disclosed in U.S. Pat. No. 6,438,408 issued to Mulligan et al., hereby incorporated herein by reference in their entirety, and the above incorporated U.S. Pat. No. 5,987,352 issued to Klein et al.

Patient alert triggering conditions may be defined according to criteria applied to one or more monitored HF signals, individually or in combination. For example, if a monitored signal or parameter derived from a monitored signal crosses a predefined threshold, a patient alert trigger 92 may be generated. Patient alert triggering conditions may be predefined according to historical clinical experience or may be programmed by a clinician according to individual patient need. Upon detecting a patient alert triggering condition, a patient alert display is generated by the patient communicator 20 based on the patient alert data received from IMD 10. The patient alert condition displayed may be generally indicated as a HF status condition or may be indicated more specifically according to the physiological change detected, for example as a low or high blood pressure. The severity or urgency of the condition may be indicated which may correspond to a monitored signal or derived parameter crossing different threshold levels. In accordance with the triggering condition and its severity, the appropriate medical personnel contact information and any additional patient instructions are displayed.

Edema monitoring 86 typically includes monitoring an impedance signal for detecting a change in thoracic impedance relating to a change in lung wetness, which may be discerned through evaluation of a change in respiration. Edema monitoring methods may be embodied as generally disclosed, for example, in U.S. Pat. No. 6,512,949 issued to Combs, et al., hereby incorporated herein by reference in its entirety. Other methods known for use in implantable medical devices for detecting changes in lung wetness may be substituted. A change in an edema monitoring signal or derived parameter that crosses a predefined threshold may trigger a patient alert signal 92, causing transmission of patient alert data to the patient communicator 20 and a subsequent display of patient alert information. The patient alert type may be identified on display 76 as lung wetness related change, or more specifically as volume overload or over-diuresis. The recommended action will typically include the contact number for a heart failure nurse and may further include adjustments to prescribed medications such as diuretics.

Arrhythmia monitoring 88 may be performed for detecting and discriminating a number of arrhythmias, including both atrial and ventricular arrhythmias. Methods for detecting and discriminating cardiac arrhythmias are well known in the art and may include evaluation of cardiac event intervals, interval patterns and EGM signal morphology. Examples of arrhythmia detection and discrimination methods are generally disclosed in U.S. Pat. No. 6,141,581 issued to Olson, et al., and in U.S. Pat. No. 6,393,316 issued to Gillberg et al., both of which patents are incorporated herein by reference in their entirety. Patient alert conditions may be defined relating to the detection of one or more types of arrhythmias and/or the impending need for delivering an arrhythmia therapy. A patient communicator display of patient alert information may indicate generally an arrhythmia condition or may specify the type of arrhythmia detected and any therapy to be or already delivered.

Patient alert information displayed may further recommend patient actions, such as preparing for an impending arrhythmia therapy, contacting an electrophysiology nurse using contact information provided on display 76, contacting emergency responders, or initiating a remote monitoring session. Recommended patient actions may also include adjustments to prescribed medications, such as beta-blockers or other anti-arrhythmia medications, in response to certain arrhythmia alert conditions such as a detected level of atrial fibrillation burden or rapid atrial-ventricular conduction. In some embodiments, patient communicator 20 may be enabled to automatically telephone or alert emergency responders. A patient may be debilitated by a life-threatening condition such as ventricular fibrillation, in which case the patient communicator 20 may be used to alert emergency responders automatically using communication module 79 to place a phone call or send and electronic message.

Device performance monitoring 90 shown in FIG. 3 may include monitoring predicted battery life, cardiac pacing thresholds, rate of pacing capture success, frequency of cardiac pacing, frequency of arrhythmia therapies, or other therapy delivery frequency or other device diagnostic testing. Patient alert triggering conditions associated with device performance parameters may be predefined or programmed in by a clinician. Patient alert information displayed by the patient communicator 20 in response to a patient alert may indicate generally a device performance condition or more specifically the type of device performance condition that caused the patient alert to be triggered. The urgency of the triggering condition may also be indicated. Recommended action by the patient, including appropriate medical personnel contact information will be displayed.

FIG. 5 is a flow chart summarizing steps included in a patient alert method. At initial step 205 of method 200, the patient alert trigger conditions are set. Patient alert trigger conditions may be set corresponding to any monitoring or self-diagnostic functions available in the associated IMD. In some cases, not all available monitoring or self-diagnostic functions will be enabled or, if enabled, may not be selected as conditions for which patient alerts are triggered. Selected patient alert trigger conditions may be predefined according to nominal condition criteria or may be programmed into the IMD by a clinician based on individual patient need. Thus, in some embodiments, a clinician may be able select which monitoring or diagnostic functions are enabled for triggering a patient alert and may additionally be able to set the conditions or criteria (e.g., signal threshold levels or ranges) under which the particular monitoring or diagnostic function causes a patient alert trigger.

After setting the trigger conditions, the IMD operates normally at step 210, performing any enabled monitoring or diagnostic functions in accordance with normal device operation. Typically the IMD will perform a comparative analysis on a periodic or continuous basis to determine if a sensed signal or derived parameter satisfies predefined alert trigger conditions. If a patient alert trigger condition is detected as determined at decision step 215, a patient alert signal is generated causing patient alert data to be transmitted to the external patient communicator from the IMD at step 220. In some embodiments, patient alert data is transmitted automatically, without patient intervention. In other embodiments, a sensory patient alarm is delivered by the IMD to notify the patient that a patient alert condition has been detected. The patient, upon sensing an alarm from the IMD, may then manually initiate a telemetry transmission from the IMD to the patient communicator. In still other embodiments, patient alert data may be stored by the IMD until an interrogation session is performed by the patient using the patient communicator or a home monitor incorporating the patient communicator. A patient may be advised to perform an interrogation session on a periodic basis in order to obtain any patient alert information.

The patient communicator generates a display of patient alert information at step 225 after receiving the patient alert data from the IMD. The patient alert information may be displayed as described previously in conjunction with the display fields shown in FIG. 4.

At optional step 230, patient alert data may be forwarded by the patient communicator or a home monitor to a clinic or clinician by transferring the data to a central database via an appropriate communication link. Step 230 may occur automatically after the external patient communicator or home monitoring unit has received patient alert data, during the next scheduled remote patient monitoring session, or may require the patient to manually initiate a transfer of the patient alert information to a central database.

In the case of an urgent, particularly life-threatening alert condition, the patient communicator may be enabled to automatically contact emergency responders as indicated at optional step 235. For example, the patient communicator may be enabled to place a “911” telephone call and play a recorded message indicating the location and condition of the patient or otherwise send a signal or communication to an emergency dispatcher.

Thus an IMD system and associated patient alert method have been described. The embodiments described herein illustrate the various aspects of the present invention and how they may be practiced. It is recognized that one having skill in the art and the benefit of the teachings provided herein may conceive of numerous variations for implementing the various aspects of an IMD system having a patient alert as disclosed herein. As such, the illustrated and described embodiments should be considered exemplary, not limiting, with regard to the following claims.

What is claimed is:

1. A method for use in an implantable medical device system, comprising:
storing a number of predefined patient alert conditions and corresponding patient instructions;
sensing signals to detect the predefined patient alert conditions;
transmitting a patient alert signal to an external device upon detecting a predefined patient alert condition; and
displaying a patient alert message on the external device to communicate the patient alert condition detected and corresponding patient instructions to the patient.

2. The method of claim 1 wherein, the patient instructions include medical personnel contact information.

3. The method of claim 1 further including displaying an indication of the urgency of the detected patient alert condition.

4. The method of claim 1 further including transmitting a communication to a medical care provider, emergency responder or other third party.

5. The method of claim 1 further including transmitting patient alert data to a centralized database used for remote patient monitoring.

6. The method of claim 1 further including generating a sensory patient alarm upon detecting a patient alert condition to notify the patient that a patient alert signal is ready for transmission to the external device.

7. An implantable medical device system, comprising:
one or more sensors for monitoring signals used in detecting a plurality of predefined patient alert conditions;
an operating system included in an implantable medical device for detecting the plurality of predefined patient alert conditions based on the sensor signals and for generating a patient alert signal upon detecting a patient alert condition;
wireless communication circuitry for transferring the patient alert signal from the implantable medical device to an external device;
a memory for storing a set of patient instructions corresponding to each of the plurality of predefined patient alert conditions; and
a display included in the external device for displaying a patient alert message in response to the external device receiving the patient alert signal wherein the patient alert message includes an indication of the detected patient alert condition and the corresponding patient instructions.

8. The system of claim 7 wherein the external device is a home monitor or programmer.

9. The system of claim 7 wherein the wireless communication circuitry is a long-range telemetry system that enables transfer of the patient alert signal from the implantable medical device to the external device without patient intervention.

10. The system of claim 7 further including a sensory patient alarm unit for notifying the patient of a pending or already transferred patient alert signal.

11. An implantable medical device system, comprising:
means for sensing signals for detecting a plurality of predefined patient alert conditions;
means for storing patient instructions corresponding to each of the predefined patient alert conditions;
means for generating a patient alert signal upon detecting a patient alert condition;
means for transferring the patient alert signal to an external display means wherein the external display means generates a patient alert message in response to the patient alert signal, the patient alert message including the patient alert condition detected and corresponding patient instructions.

12. A computer-readable medium containing instructions that when implemented in an implantable medical device system cause the system to:
detect a plurality of predefined patient alert conditions;
generate a patient alert signal upon detecting a predefined patient alert condition;
display a patient alert message in response to the patient alert signal wherein the message includes an indication of the detected patient alert condition and corresponding patient instructions.

13. The computer-readable medium of claim 12, wherein the patient alert message further includes an indication of the urgency of the detected patient alert condition.

14. The computer-readable medium of claim 12, further causing the system to generate a sensory patient alarm upon detecting a patient alert condition.

15. The computer-readable medium of claim 12, further causing the system to automatically transfer patient alert data to a central database.

16. The computer-readable medium of claim 12, further causing the system to automatically transmit a communication to medical personnel, an emergency responder, or other third party.

17. An external medical device, comprising:
wireless communication circuitry for receiving a patient alert signal from an implantable medical device;
an operating system for generating a patient alert message in response to receiving the patient alert signal; and
a display for displaying the patient alert message.

18. The external device of claim 17 further including a speaker for broadcasting audible sounds to alert a patient of the presence of a patient alert message.

19. The external medical device of claim 17 wherein the patient alert message display includes patient instructions.

20. The external device of claim 19 wherein the patient instructions include medical personnel contact information.

21. The external device of claim 17 further including a communication link to a central database for transferring patient alert data to the central database.

22. The external device of claim 17 further including memory for storing information to be displayed in the patient alert message.

23. The external device of claim 22 wherein the memory is programmable.

24. The external device of claim 23 wherein the memory is programmable using a centralized programming system.

25. The external device of claim 17 further including a communication module for transmitting a telephone or electronic communication in response to a patient alert signal.

26. The external device of claim 17 further including a user interface.

27. The external device of claim 17 wherein the device is adapted to be worn by a patient.

28. The external device of claim 27 further including a global positioning module for use in locating the global position of a patient.
